(19) United States
(12) Patent Application Publication
(10) Pub. No.: US 2015/0133666 A1
(43) Pub. Date: May 14, 2015

(54) TRITOQUALINE FOR USE IN THE TREATMENT OF CYSTIC FIBROSIS

(71) Applicant: Gaetan Terrasse, Montceau Les Mines (FR)

(72) Inventor: Gaetan Terrasse, Montceau Les Mines (FR)

(21) Appl. No.: 14/403,484

(22) PCT Filed: May 21, 2013
(86) PCT No.: PCT/FR2013/051101

§ 371 Date: Nov. 24, 2014

(30) Foreign Application Priority Data
May 24, 2012 (FR) 1201482

(51) Int. Cl. A61K 31/4355

(52) U.S. Cl. CPC A61K 31/4355
USPC 546/90

(57) ABSTRACT

The present invention relates to the use of a histamine H4 agonist molecule, the enantiomers of the (AMINO-7 TRIETHOXY-4,5,6 OXO-1 DIHYDRO-1,3 ISOBENZOFURANNYL-3)-1METHOXY-8 METHYL-2 METHYLENEDIOXY-6,7 TETRAHYDRO-,2,3,4 ISOQUINOLINE or tritoqualine for the treatment of respiratory impairment caused by cystic fibrosis and the reduction and prevention of bronchial superinfections.

The present invention relates to the use of chemicals, the levorotatory and dextrorotatory enantiomers of the (AMINO-7 TRIETHOXY-4,5,6 OXO-1 DIHYDRO-1,3 ISOBENZOFURANNYL-3)-1METHOXY-8 METHYL-2METHYLENEDIOXY-6,7TETRAHYDRO-,2,3,4ISOQUINOLEINE or tritoqualine to treat respiratory deficits and infections caused by cystic fibrosis.

The French mucoviscidose (meaning “disease of the viscous mucus”), or the English cystic fibrosis (implying “of the pancreas”), is a genetic disease affecting the glandular epithelium of many organs.

It is the lethal genetic disease with autosomal recessive transmission the most common in populations of Europoid type, while it is very rare in African and Asian populations.

It is linked to mutations in the CFTR gene on chromosome 7, resulting in an alteration of the CFTR (acronym for Cystic Fibrosis Transmembrane conductance Regulator) protein. This protein is an ion channel permeable to chlorine, to thiocyanate, whose function is to regulate the transport of chlorine across cell membranes.

Its dysfunction causes an increase in mucus viscosity and its accumulation in the respiratory and digestive tracts. The disease affects many organs but respiratory diseases are prevalent and represent the bulk of morbidity.

The respiratory impairment results in a progressive decline of FEV1 (Forced Expiratory Volume in 1 second) which turns into a chronic respiratory failure.

The lungs are also the target of chronic infections, due to the change of mucus goblet cells of the lungs.

These are the infections and bronchial congestion that lead ultimately to respiratory distress, often the cause of death.

In France, one newborn in 4200 is affected by cystic fibrosis. Indeed, this genetic disease is quite common. Thus, 2 million people carry the gene and can pass it to their children. Every year, about 200 children are born in France with cystic fibrosis.

There is no cure, but advances in treatment have helped improve the quality of life and life expectancy of patients; thus, in France, life expectancy at birth increased from 7 years in 1965 to 47 in 2005.

There are approximately 70,000 patients in Europe and the USA and it is in these areas that the pathology is the most important. In Asia, the prevalence is very low or non-existent, as in Japan. The prevalence of the disease reveals significant variations thereof (from 1 to 10): from 1 in 2500 for the Europeans to 1 in 32,000 for the Asians.

The biological diagnosis is based on the sweat test confirmed by identification of genetic mutations. Neonatal screening, widespread in France since 2002, allows diagnosis and early treatment.

Cystic fibrosis, scientifically described as a disease in 1936, was in fact already long known. In the Middle Ages, people knew about the disastrous fate of a newborn whose mother had noticed the “salty kiss”, that is to say, the salty taste left by a kiss on the forehead of the child.

In the early twentieth century, the first observations appeared on the association of pulmonary disease, diarrhea and pancreatic anomaly with multiple cases in the same family. In 1912, Garrod described families whose children had a fatty diarrhea and died from lung infection.

It was in 1936, in a thesis written in German and chaired by the Swiss pediatrician Guido Fanconi, that the disease was described for the first time in children with suspected celiac disease, under the name of “cystic pancreatic fibrosis and bronchiectasis”.

Cystic fibrosis was not regarded as a distinct pathological entity until 1938 by the American pediatrician Dorothy Hansine Andersen, a physician at the Babies' Hospital of New York, who published an article entitled “Cystic fibrosis of the pancreas and its relationship with celiac disease”.

In 1989, the gene involved in cystic fibrosis was isolated by the teams of Lap-Chi Tsui, Collins and Riordan. The genetic anomaly responsible for the disease was finally discovered. It is a mutation of a gene located at 7q31 and containing 27 exons, named Cystic Fibrosis (CF) encoding a transmembrane protein called Cystic Fibrosis transmembrane conductance Regulator (CFTR) composed of 1480 amino acids. It was only a little later that evidence was brought that CFTR was indeed a chlorine channel.

The discovery of the genetic defect subsequently allowed adding diagnostic genotyping protocol and considering gene therapy.

In the 1940s, the disease was considered primarily as a nutritional problem with a deficiency in vitamin A. The management essentially consisted of a high protein diet, intramuscular injections of vitamin A in high doses, pancreatic extracts and penicillin inhalations.

In 1945, Dorothy Andersen recommended “a diet low in fat, high in protein, with a free proportion of vegetables, fruits and sugars and a moderate restriction of starches.

The first antibacterial drug, a sulfonamide marketed under the name of Prontosil, was available in 1934 and penicillin in injectable form in 1944. Other antibiotics followed and had a key role in the treatment of patients.

In the late fifties, the main pathogenic germ was Staphylococcus aureus and many strains were still susceptible to penicillin. During that decade, other antibiotics appeared. As early as 1951, while Staphylococcus aureus was the bacterium usually found, an increase in the frequency of Pseudomonas aeruginosa was observed, attributed to prolonged antibiotic treatments, however the benefit of aggressive antibiotic treatments became progressively evident.

Numerous therapeutic classes are currently used in cystic fibrosis, but the results do not always meet the expectations.

Gene therapy: Cystic fibrosis being a monogenic disease, which is to say involving only one gene, it is natural that high hopes of healing are born with the appearance of the concept of gene therapy.

Analyses of the amount of the RNA messenger (mRNA) in healthy cells showed an extremely low number of mRNA encoding CFTR, between two and three copies per cell. In theory, even with a very low transfer rate, it should be possible to restore a normal secretion function in lung cells by bringing one or two copies of the healthy gene integrated into a vector.

Once the function is restored, the mucus should become more fluid and allow a satisfactory mucociliary clearance. Indeed, an infection of the pulmonary tract by pathogens and the inflammation that follows is one of the causes of loss of respiratory function.

But despite the emergence of gene therapy, the results have so far given no conclusive result.

The Copernicus Therapeutics Company has used compacted DNA (non-viral) to introduce it via inhalation. A Phase I clinical trial demonstrated changes in the nasal mucosa but did not demonstrate a better expression of the gene introduced via inhalation.

The modulation of the expression of CFTR: Many companies have molecules in development, in particular the VERTEX Company.

The latter recently launched in 2012 a molecule called Kalydeco which improves the respiratory function, albeit not significantly (10% improvement in FEV1).

Two other products are also under development, the Ataluren and the VX 809, with also limited clinical results on respiratory function.

The anti-infective products: Such infections are acute or chronic bronchial superinfections that will gradually damage respiratory function. The main germs are Staphylococcus aureus, Pseudomonas aeruginosa and Burkholderia cepacia. Most of these germs are resistant to antibiotics such as penicillin.

Colimycin-based and tobramycin-based antibiotics are mainly used. The products are inhaled, either in dry form (Tobi Podhaler, Colobreathe) or wet form (Tadim, Tobi, Cayston, colimycin).

Other products are also being evaluated: Azithromycin from Pfizer—in patients chronically infected with Pseudomonas aeruginosa, this antibiotic used via inhalation allows a net weight gain and fewer hospitalizations.

Aztreonam from Gilead Sciences is an antibiotic in inhaled form which received FDA approval on Feb. 22, 2010 and is available to US patients.

There are also several other products in development such as Arikace, a liposomal formulation of amikacin, but also KB001 from KaloBios Pharmaceuticals which initiated a Phase I clinical trial to test the safety of their approach to the treatment by antibody of infections caused by Pseudomonas aeruginosa.

Other antibiotics are also being developed to treat infections of cystic fibrosis such as DMP-376 which is a new formulation of levofloxacin, and GS 9310/11 from Gilead Sciences, which is a combination of fosfomycin and tobramycin administered by inhaled route.

The path of anti-inflammatory drugs has also been explored such as ibuprofen, N-acetylcysteine, Docosahexaenoic acid (DHA), the humanized monoclonal antibody KB001, GSK SB 656 933 and Sildenafil.

In this class, only ibuprofen demonstrated its effectiveness at a low cost but the results obtained are on a small scale.

The products used in chronic bronchitis and asthma did not work.

Yet, there is great potential in this therapeutic class, because cystic fibrosis is primarily an inflammatory disease of the lung and gastrointestinal tract.

Mucus modifiers are also a therapeutic class in development. In cystic fibrosis, changes in the transport of salt in the cells make the mucus very thick and sticky. This approach targets proteins other than the CFTR protein to improve the flow of salt in and out of cells, allowing the mucus to be more hydrated and, therefore, more easily eliminated.

Many products are in use or under development such as a hypertonic saline solution, the Denufosol from Inspire Pharmaceuticals, which allows correcting the ion transport defect in lung cells. This product increases the FEV1 compared to the placebo. There are also the Bronchitol (Mannitol) from Pharmaxis which theoretically should help rehydrate lung secretions.

In this class, there are 3 other products under development, the SPI-8811 from Sucampo Pharmaceuticals, and also the 1901 from the Lantibio Company, as well as the Gilead GS9411 which works by blocking the absorption of sodium.

In this class of products, the hyper saline solution is already on the market with over 10,000 treated patients. The cost of treatment is low and in direct competition with new products in development at Pharmaxis (Bronchitol) and at GSK (GSK9411), whose results on the FEV1 are not above that of the hyper saline solution.

The alteration of the mucus is also currently being treated by Pulmozyme from Genentech on over 18,000 patients worldwide.

Despite numerous therapeutic classes used, while the survival of patients with cystic fibrosis has increased, treatment outcome is still very inadequate.

Tritoqualine is a chemical substance that has been known for very many years and used as antihistamine. Its manufacture is described in French Patent FR 1,295,309.

Tritoqualine is 7-Amino-4,5,6-triethoxy-3-(5,6,7,8-tetrahydro-4-methoxy-6-methyl-1,3-dioxolo[4,5-g]isoquinolin-5-yl) phthalide. In its marketed pharmaceutical form it is in the form of a mixture of enantiomers.

Tritoqualine is known for its antiallergic activity by its inhibitory action on histidine decarboxylase. This activity is however very low and does not explain the many properties that it has on various clinical symptoms such as rhinitis, urticaria, eczema and mastocytosis.

The inventor, together with other inventors, has shown that tritoqualine had a very important effect on a new receptor, the histamine H4 receptor.

This activity of tritoqualine on the H4 receptor has been demonstrated in a US Patent (US2010144718A1) “TREATMENT OF DISEASES MODULATED BY A H4 RECEPTOR AGONIST”, Gaëtan Terrasse et al., recently accepted.

However, this patent does not describe the activity of tritoqualine on cystic fibrosis.

Another patent on H4 agonists describes the use of these products in the protection of hematopoietic precursors in the context of chemotherapy: WO2008006974A2 “Use of histamine H4 receptor ligands to protect hematopoietic progenitors against the hematological toxicity of chemotherapeutic agents”, Michel Dy et al.

Neither that patent, nor any other patent or scientific paper, highlights the action of H4 agonists to histamine in cystic fibrosis.

Commercial tritoqualine presents itself in the form of a white powder, very sensitive to light which degrades it into cotarnine and phthalic acid.

Commercial tritoqualine (called Hypostamine) is in the form of a tablet with 100 mg concentration per tablet.

Tritoqualine has 2 asymmetric carbons, but the commercial form is a mixture of 2 enantiomers.

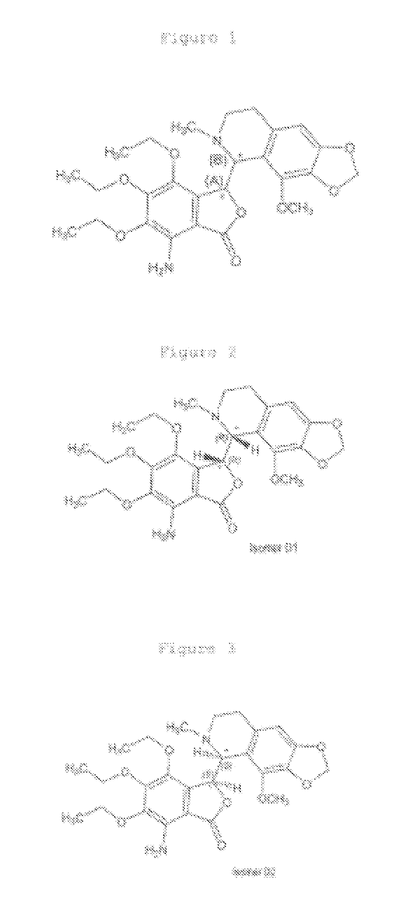

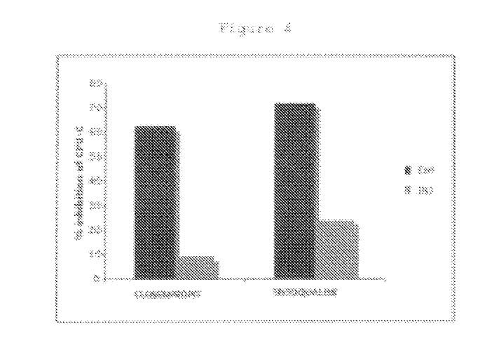

FIG. 1 illustrates the presence of asymmetric carbons, which are labeled A and B. FIG. 2 illustrates the form of the isomer D1, and FIG. 3 illustrates the form of the isomer D2.

FIG. 4 compares tritoqualine and Clobenpropit, the H4 agonist of reference. This figure highlights that tritoqualine is capable of inhibiting the proliferation of CFU rich in H4 receptor. When using an anti H4, tritoqualine activity is greatly reduced, as is that of Clobenpropit. This clearly demonstrates that tritoqualine is a H4 agonist molecule.

The surprising properties of commercial tritoqualine on cystic fibrosis were identified.

Tritoqualine is used at a dose from 100 mg to 800 mg, and preferably at a dose from 200 to 400 mg, in the following examples.

### EXAMPLE 1

It is the case of a 10-year old child suffering from cystic fibrosis who had a significant respiratory impairment. During the initial consultation, a treatment based on tritoqualine was started at a daily dose of 200 mg.

Before therapy with tritoqualine, the FEV1 (Forced Expiratory Volume in 1 second) of this young patient was 0.39 L/s or 55.71% of the normal expected value (based on age, sex, and size). The flow rate was measured with a Jaeger® MasterScreen® Body plethysmography apparatus.

This level of ventilatory flow signals a severe respiratory impairment. This patient also had 15 lung infections per year prior to treatment. After 6 months of treatment with tritoqualine at a dose of 200 mg/day, the ventilatory flows were again measured. At this second visit, the FEV1 parameter was measured at 0.50 L/s. That was an increase of 28.2% compared to the initial flows measured during the first visit. During the third visit, months after the first visit, the flows were again measured. FEV1 was then 0.62 L/s, an improvement of 58.7% compared to the initial rates.

Over 12 months, the number of superinfections decreased from 15 to only 4 infections. Thus, surprisingly, tritoqualine increased the ventilatory flows of this young patient by more than 50% and divided by three the number of infections.

### EXAMPLE 2

An 18-year old adult with cystic fibrosis was seen in consultation for the first time during which treatment worth tritoqualine was started at a dose of 400 mg per day. Before therapy with tritoqualine, the FEV1 of the patient was measured at 0.45 L/s or only 69.23% of the normal expected value (based on age, sex, and size).

This patient had had 8 lung infections per year prior to treatment. After 6 months of treatment at the daily dose of 400 mg of tritoqualine, the FEV1 parameter was measured again during a second consultation. The FEV1 parameter measured during this consultation went from 0.45 L/s to 0.50 L/s, or an improvement of 11.11% of the ventilatory flows.

A third consultation after 12 months of treatment showed that the FEV parameter went from 0.50 L/s to 0.58 L/s. Compared to the initial visit, that increase represented a 28.89% increase in FEV1 (100*(0.58-0.45)/0.45 over baseline).

During the 12-month treatment with tritoqualine (400 mg daily), the patient benefited from a significant reduction in pulmonary infections, from 8 initially to only 3 infections after 12 months of treatment.

This decrease in infections and increase in ventilation flows are quite surprising, especially as the conventional treatments of this patient had no effect on his respiratory function.

### EXAMPLE 3

A 12-year old child with cystic fibrosis was treated during his first consultation with a dose of 300 mg of tritoqualine.

Before therapy with tritoqualine, the FEV1 of the patient was measured at 0.70 L/s, or 63.64% of the normal expected value (based on age, sex, and size). This patient had had 12 pulmonary infections per year before the treatment. After a 6-month treatment with a daily dose of 300 mg of tritoqualine, the FEV1 parameter was measured at his second consultation at 1.0 L/s. This represents an improvement of 42.86% over the initial visit. (100*(1.00 at 0. 0.70)/0.70). After 12 months of treatment, the FEV1 parameter measured during the third consultation was 1.20 L/s or 71.43% improvement compared to the initial visit. (100*(1.20-0.70)/0.70).

During the 12-month treatment with tritoqualine at 300 mg per day, this patient had a significant reduction in pulmonary infections. These decreased from 12 to 3 infections in one single year.

It is thus evident that tritoqualine surprisingly reduces the number of superinfections in patients and increases ventilatory flows in parallel. Moreover, tritoqualine has a particularly important effect on ventilatory flows, far superior to all the existing treatments to date. We can therefore say that tritoqualine, in its classical form of a mixture of enantiomers at a dose of 100-400 mg, can treat the respiratory degradation of cystic fibrosis. The treatment also significantly reduces bronchial superinfections as well as hospitalizations.

Tritoqualine could be used in different forms, apart from its tablet form, without changing its effectiveness, such as in the forms of capsules, syrup, oral gel or controlled-release tablets.

1. A substance having an agonist activity on the histamine H4 receptor for the treatment of cystic fibrosis, characterized in that it comprises an isomer or a mixture of isomers of 7-Amino-4,5,6-triethoxy-3-(5,6,7,8-tetrahydro-4-methoxy-6-methyl-1,3-dioxolo[4,5-g]isoquinolin-5-yl) phthalide, or tritoqualine.

2. Substance according to claim 1, characterized in that it is presented in various pharmaceutical forms such as tablets, capsules, syrup and gel.
